(19) United States
(12) Patent Application Publication
(10) Pub. No.: US 2006/0057537 A1
(43) Pub. Date: Mar. 16, 2006

(54) Combination dental instrument

(75) Inventors: Richard A. Tamburrino, Auburn, NY (US); Roger W. Leseberg, Syracuse, NY (US); Michael T. McMahon, Syracuse, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc., Skaneateles Falls, NY (US)

(21) Appl. No.: 11/095,461

(22) Filed: Mar. 30, 2005

Related U.S. Application Data
(63) Continuation-in-part of application No. 10/941,530, filed on Sep. 15, 2004.
(63) Continuation-in-part of application No. 10/941,280, filed on Sep. 15, 2004.

(51) Int. Cl. A61B 1/24; A61C 3/00

(52) U.S. Cl. 433/30; 433/29

(57) ABSTRACT

A dental instrument includes a handle and a first light generating system releasably contained in the handle. The first light generating system includes a first light source, a first dental appliance releasably attachable to the handle and coupled to the first light generating system, a second light generating system which can be substituted into the handle for the first light generating system. The second light generating system includes a second light source which is different than the first light source. The instrument is battery powered, wherein each of the light generating systems is configured mechanically to prevent reverse battery polarity.

### CROSS REFERENCE TO RELATED APPLICATIONS

This patent application is a continuation-in-part application of commonly assigned and co-pending U.S. Ser. No. 10/941,530 entitled ILLUMINATED DENTAL EXAMINATION INSTRUMENT and U.S. Ser. No. 10/941,280 entitled CORDLESS INTRAORAL DENTAL EXAMINATION INSTRUMENT HAVING NON-PLANO MIRROR, each filed on Sep. 15, 2004, the entire contents of each being incorporated in their entirety by reference.

### FIELD OF THE INVENTION

This invention relates to the field of medical diagnostic instruments and more particularly to an instrument that can interchangeably be used for performing multiple dental procedures such as, for example, examining a patient, photo-curing a dental composition, and/or teeth bleaching.

### BACKGROUND OF THE INVENTION

A number of instruments are used in the typical dentist's office for performing or assisting in the performance of a number of varied procedures relating to a patient. For example, intraoral dental mirrors are repletely known as an extremely convenient apparatus for aiding in the inspection of the teeth of a patient. Typically, these instruments include an elongated body or handle having a retroflexed mirrored portion that permits inspection of both the upper and lower arches of teeth. Illuminated versions of these devices typically include a halogen lamp or other similar miniature light source, such as those that are described in U.S. Pat. Nos. 5,741,132 and 6,575,744B1, the light source providing sufficient illumination so as to enable or enhance visualization of the teeth using the mirror. In each of the preceding, the light source is provided beneath the mirror in a retroflexed portion of the instrument. Alternatively, as described, for example, in U.S. Pat. Nos. 5,139,421 and 5,457,611, to Verderber, a miniature halogen or other incandescent bulb is retained within the handle of the instrument that is optically coupled to a light guide having the mirror portion, the guide being releasably attached to the distal end of the instrument.

One problem encountered in the use of incandescent bulbs as an illumination source in instruments, such as those described above, relates to heat generation developing from the contained bulb, thereby requiring the placement of suitable heat sinks or other heat dissipation means in the instrument and/or the use of open slotted vents in the gripping handle thereof. These solutions to the above problem complicate the overall design and manufacture of the instrument, including the cost thereof.

It has been considered with evolving technological advances to possibly utilize light emitting diodes (LEDs) as a light source in lieu of miniature halogen bulbs. Overall, LEDs are less sensitive to shock damage than incandescent bulbs or lamps, have an increased overall life, generate less heat, are more efficient, and are available in a wider range of color temperatures.

Curing lamps such as described, for example, in U.S. Pat. No. 5,634,711, are another dedicated instrument used in the field of dentistry that is used in connection with photocurable dental compositions. These devices also use a light source, though the light emitted is typically a blue light having a wavelength of approximately 420 to 500 nanometers to permit a photochemical reaction to occur when the light is made incident upon the composition to which the light source is aimed. Though tungsten and other lamps have been utilized, the incorporation of blue LEDs has become more evident as described in U.S. Patent Application Publication 2003/0081430. To date, however, there has been no significant effort to combine the features of the heretofore separate and distinct devices in order to provide greater versatility in the dentist's office.

It is a general prevailing need in the field to provide savings in terms of cost and time in the performance of patient visits.

### SUMMARY OF THE INVENTION

Therefore and according to one aspect of the present invention, there is provided a dental instrument comprising:
a handle;
a first light generating system releasably contained in said handle, said first light generating system including a first light source;
a first dental appliance releasably attachable to said handle and coupled to said first light generating system; and
a second light generating system which can be interchangeably substituted in said handle for said first light generating system, said second light generating system including a second light source which is different than the first light source.

Preferably, each of the first and second light generating systems include at least one LED. According to one version, the first light generating system includes at least one white LED wherein the first dental appliance is an intraoral mirror. According to one version, the intraoral mirror is disposable. The second light generating system includes at least one blue LED for curing a dental composition wherein a separate or second dental appliance, such as a transilluminator, is selectively coupled to the handle and the second light generating system. According to yet another version, the second or other dental appliance can be used for dental bleaching or whitening or other procedures requiring a light source to either perform or assist in the performance of the procedure.

According to one version, each of the light generating systems can be housed within individual illumination cartridge assemblies that can be selectively placed into a cavity or compartment of the handle. Each illumination cartridge assembly commonly includes a body or housing that retains at least one type of LED and circuitry, permitting electrical engagement relative to a switch assembly and a power supply of the instrument, such as contained batteries. Each illumination cartridge assembly, according to the present invention can be removed from the confines of the handle, along with the dental appliance(s) in order to permit versatility using a single instrument.

According to another preferred aspect of the present invention, there is provided a method for performing separate dental procedures on a patient, said method comprising the steps of: performing a first dental procedure on said patient using a dental instrument, said instrument having a first light source contained therein suitable for said first procedure; releasably interchanging a second light source for said first light source in said instrument; and performing a second dental procedure on said patient using said instrument.

In one version, the batteries are stored within a casing in a stacked relationship relative to the cartridge. The cartridge is configured so as to prevent reverse battery polarity through unwitting or incorrect placement of the batteries, thereby permitting the resident circuitry to be made compactly and without requiring additional circuit components.

An advantage of the present invention is that a single instrument can be used in a dentist's office to effectively perform at least two varied procedures. As a result, patient inconvenience is reduced and the time of visits can be more effectively conducted.

Yet another advantage provided by the present invention is that the present combinational dental instrument can be cordless, and is therefore quite convenient for the caregiver (e.g., dentist or hygienist) to utilize, the instrument preferably being conveniently powered by a set of rechargeable batteries.

Still another advantage of the present invention is that less heat is generated using at least one LED as an illumination source rather than an incandescent bulb while producing sufficient illumination for examination purposes.

Still another advantage of the present instrument is that it can be easily disassembled so that the batteries and illumination housing can be set aside, allowing the handle portions can be autoclaved (e.g., sterilized).

Yet another advantage of the present invention is that the herein described instrument can be used in the field without the risk that the batteries will be used or replaced incorrectly, so as to cause potential damage to the resident circuitry due to reverse battery polarity.

These and other objects, features and advantages of the present invention will become apparent from the following Detailed Description.

### BRIEF DESCRIPTION OF THE DRAWINGS

FIG. 1 is a perspective view of a combinational dental instrument made in accordance with an embodiment of the present invention;

FIG. 2 is a partial exploded view of the instrument according to FIG. 1;

FIG. 3 is an exploded view of a light generating system for the instrument of FIGS. 1 and 2;

FIG. 4 is a perspective view of the instrument of FIGS. 1-3 configured for an examination mode;

FIG. 5 is a perspective view of the instrument of FIGS. 1-3 configured for a photo-curing mode;

FIG. 6 is a bottom perspective view of the illumination cartridge of FIG. 3;

FIG. 7 is a partial side elevational view, partly in section, of the illumination cartridge of FIG. 6, depicting proper mechanical and electrical engagement with a contained battery; and

FIG. 8 is the partial side elevational view of FIG. 7, depicting an incorrect mechanical and electrical engagement between the illumination cartridge and contained battery and reverse battery polarity preventing features of the cartridge.

### DETAILED DESCRIPTION

The following description relates to a versatile combinational dental instrument that is made in accordance with an embodiment of the present invention. It will be readily apparent throughout that the instrument can be otherwise configured using the inventive concepts described herein. In addition and throughout the course of discussion that follows several terms, such as “top”, “side”, “lateral”, “bottom”, “distal”, “proximal”, “front”, “rear”, and the like are used in order to provide a convenient frame of reference with regard to the accompanying drawings. These descriptions, however, unless indicated otherwise, should not be regarded as limiting with regard to the present invention.

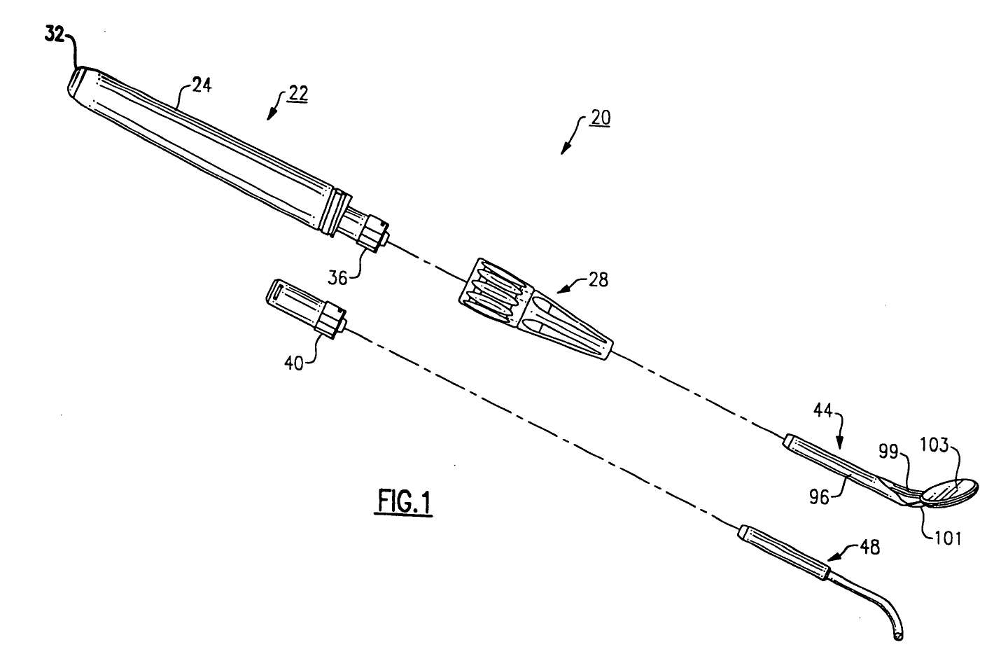

Referring now to FIG. 1, and in terms of a general overview, the combinational instrument 20 according to this embodiment includes a hollow elongated body or handle 24. A number of components are retained within the hollow handle 24, as will be described in greater detail below. For the present, however, front and rear end caps 28, 32, are used to cover the open ends 25, 26 of the elongated handle 24. The rear end cap 32 is shown in FIG. 1, as assembled.

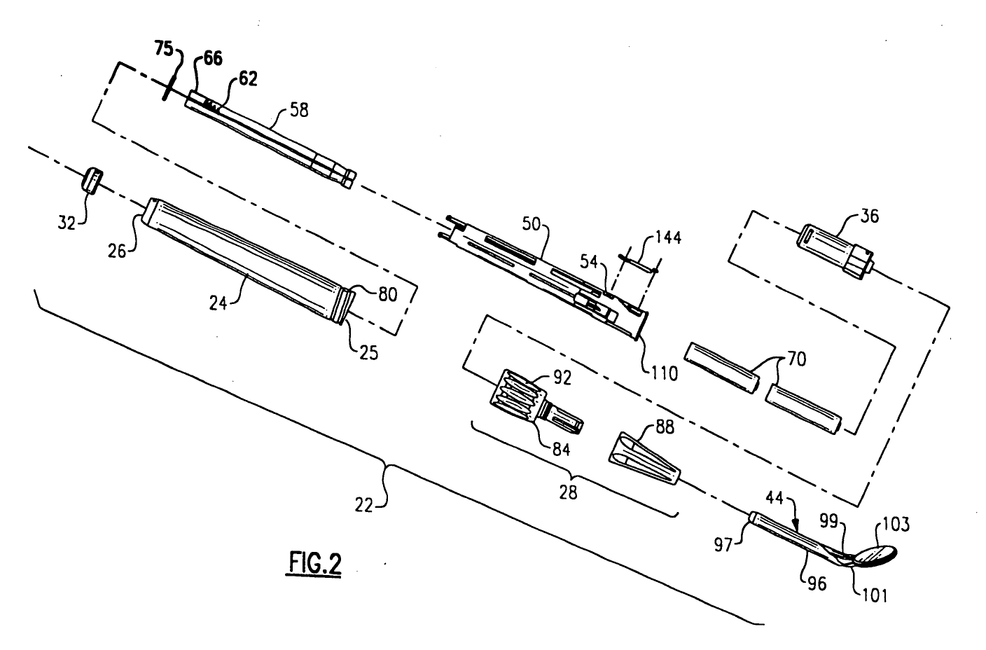

For purposes of this figure, among the components contained within the handle 24 is one of two interchangeable illumination cartridge assemblies 36, 40. One of the illumination cartridge assemblies 36 is shown as attached for purposes of FIGS. 1 and 2. A dental appliance, such as an intraoral mirror 44 or a transilluminator 48, is releasably attached to the front end cap 28 and is coupled to the light source contained within one of the illumination cartridge assemblies 36, 40. Additional details are now provided, referring to the remaining figures.

Referring to FIG. 2, an exploded view of the combination dental instrument 20 is depicted. The examination instrument 20 according to this embodiment includes a plastic molded handle 24, the handle defined herein by a cylindrical configuration and having a pair of open ends 25, 26 that further define a hollow interior which is appropriately sized for retaining a cylindrical interior cage member 50. The interior cage member 50 is preferably also made from plastic or similar insulating material which is also preferably autoclavable and includes a plurality of lateral slots 54 that are defined along the axial length thereof, the interior cage member further having means for supporting an electrically conductive battery contact strip 58, the strip including a contact spring 62 at a proximal end 66 thereof.

The herein described examination instrument 20 does not require an exterior power supply, the present instrument being cordless and powered by a set of stacked batteries 70 that are retained within a defined battery compartment that is provided within the confines of the interior cage member 50 which is also open-ended. According to this embodiment, the batteries 70 are AAA alkaline batteries but it will be readily apparent to those of sufficient skill that other types and sized batteries, such as nickel-cadmium and lithium-ion can be substituted, wherein the batteries can also preferably be rechargeable.

The contact spring 62 serves as a contact for the stacked batteries 70, relative to a disk 75 which fastens the contact spring 62 and conductive battery strip into one electrically common assembly. The disk 75 is interposed between the contact spring 62 and the rear end cap 32 that is fitted to the open proximal end 26 of the handle 24.

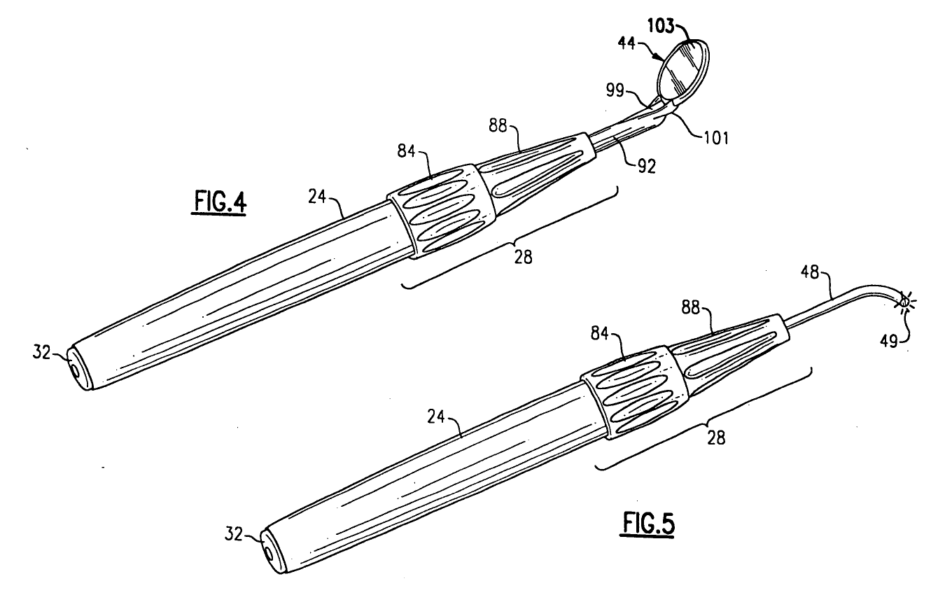

Still referring to FIG. 2, the distal end 25 of the handle of the instrument 20 includes a set of exterior screw threads 80 permitting the connection of the front end cap 28. According to this embodiment, the front end cap 28 comprises a pair of components; namely, a support collet 84 and a collet locking ring 88 wherein the end cap is used to retain one of dental appliances, in this case the disposable mirror 44 which is represented in this figure. The support collet 84 includes a rotatable exterior knob 92 that includes a set of interior threads (not shown) as well as an extending portion having a set of collets and exterior threads for receiving the collet locking ring 88, which is threadingly mounted thereupon. The disposable mirror 44 includes an elongate shank portion 96 that is engaged with the collet locking ring 88, the collet locking ring being rotated to tighten or loosen the grip on the disposable mirror 44, FIG. 4, or transilluminator 48, FIG. 5, to permit engagement or disengagement therewith.

As noted, the mirror 44 of the herein described instrument 20 includes an elongated shank portion 96. The shank portion 96 has a proximal end that includes a defined light entrance surface 97, the shank portion terminating at a heel portion having a pair of opposing substantially parallel light exit surfaces 99, 101 on each of the top and bottom sides thereof, each of which are angled relative to a primary axis that extends through the shank portion. A face portion is integrally formed at the distal end of the mirror along an axis (not shown) coextensive with that of the heel portion. The face portion of the mirror 44 according to this embodiment, is substantially circular in configuration (though other shapes could be contemplated) and includes a mirrored surface 103. The mirror 44 according to this embodiment is entirely made from a high-quality light transmissive material, such as acrylic, polyamide, polycarbonate and the like, and is preferably molded, including the face portion and the mirrored surface 103.

According to this embodiment, the mirrored surface 103 is concave in curvature in order to provide magnification of the object(s) being examined. According to this embodiment, about a 1.5× magnification is created, although this parameter can easily be varied. The reflective portion of the mirror 44 is molded according to this embodiment using “In Mold Decoration” technology wherein the contour of the mirrored surface 103 is controlled by the shape/contour of the mold. Alternatively, the mirrored surface 103 could be made with a convex or other form of curvature, wherein a minified image can be produced, thereby creating a wider field of view which can be useful for certain oral applications or according to yet another version, the mirrored surface can be flat (e.g., plans).

Additional details concerning the disposable mirror 44 are found in commonly assigned and copending U.S. Ser. No. 10/941,280, previously incorporated in its entirety by reference.

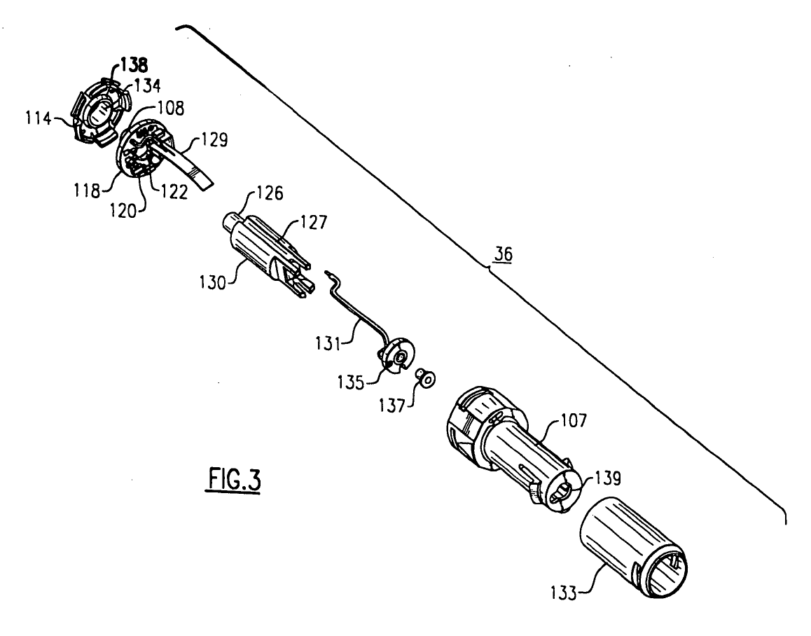

Disposed between the stacked batteries 70 and the support collet 84 of the front end cap 28 is a first light generating system that includes the first illumination cartridge assembly 36, shown more completely in FIG. 3. For purposes of this explanation, only illumination cartridge assembly 36 is discussed. A second light generating system includes illumination cartridge assembly 40 this assembly being nearly identical with those differences being related herein. The illumination cartridge assembly 36 of the described embodiment consists of a hollow cylindrical housing body 107 which is made from Radel R, polyphenylsulfone or suitable polymer, but can be fabricated from literally any electrical insulating material, wherein a miniature LED 108 (only partially shown in FIG. 3) is disposed at a distal end 110 adjacent a reflector cap portion 114 that is attached in overlaying relation thereto. In this instance, the miniature LED 108 is a Model LXHL-PW01 white LED, manufactured by Lumileds, Inc., though other similar light sources can be utilized. The illumination cartridge assembly 36 further includes a printed circuit board (PCB) 118 containing suitable electronics 120 thereupon used for driving (e.g. powering) the miniature LED 108 and for enabling the LED to be powered by a battery power source. A circuit for this purpose is described in U.S. Ser. No. 10/393,319, the contents of which are herein incorporated by reference. The PCB 118 according to this embodiment is disposed immediately below or proximal to the miniature LED 108, and includes an opening 122 for receiving the projecting portion 126 of a heat sink 130, retained by the housing body 107 in order to dissipate heat that is generated by the miniature LED 108, as well as heat also generated by the PCB 118.

The heat sink 126 includes a lateral slot 127 extending over the entire axial length thereof which is sized to retain one of a pair of electrical contacts. A first electrical contact 129 extends from the proximal side of the printed circuit board 118 and includes a lead wire and a strip member. The strip member of this electrical contact 129 is sandwiched between a cartridge collar 133 and the exterior of the cartridge body 107. A second electrical contact 131 is formed from a lead wire that extends axially from a battery contact board 135, the board being retained within the bottom of the cartridge body 107. According to this embodiment, the first electrical contact 129 is a negative electrical contact while the second electrical contact 131 is a positive electrical contact in which the positive terminal of the stacked batteries 70, FIG. 2, is contacted by a rivet 137 that is disposed within a chamfered hole 139 at the bottom of the cartridge body 107.

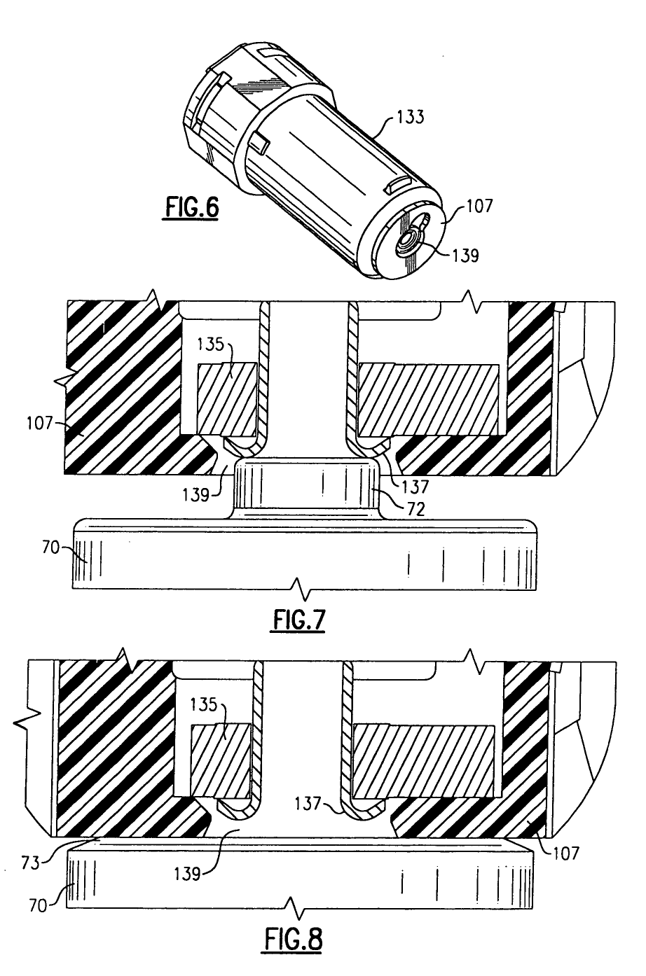

Referring to FIGS. 6-8, the rivet 137 is inset within the chamfered hole 139 of the cartridge body 107, the hole permitting the entry of the projecting positive terminal 72 of a stacked battery 70, FIG. 2, when assembled properly, to enable energization of the contained miniature LED 108, as described below. Improper positioning of the battery 70, such as shown in FIG. 8, wherein the flat negative terminal 73 is in relation with the hole 139 does not permit physical contact between the terminal and the rivet 137 and consequently no electrical connection can be made, preventing reverse polarity when the instrument is used in the field.

Still referring to FIG. 3, the reflector cap portion 114 is defined by a through opening 134 that is fitted about the lens envelope (not shown) of the miniature LED 108, the through opening preferably having an inwardly tapered surface 138 that is used in order to effectively focus the light emitted from the miniature LED 108 onto a light entering surface of the shank portion 96 of the mirror 44. The illumination cartridge assembly 36 is retained by the front end cap 28 and is held in place by the handle 24 and a contact strip of the internal cage member 50. The reflector cap portion 114 also recesses the LED lens and protects the lens from damage, such as from dropping the cartridge 36, when removed from the instrument 20 for cleaning. Additional details regarding the illumination cartridge are provided in U.S. Ser. No. 10/941,530, entitled: Illuminated Dental Examination Instrument, the entire contents of which has been previously incorporated by reference in its entirety.

The herein described dental examination instrument 20 according to this particular embodiment further utilizes a rotary switch assembly in order to selectively illuminate the miniature LED 108 that is housed in the illumination cartridge assembly 36. The rotary switch assembly is enabled by rotating the rotatable exterior knob 92 of the support collet 84 relative to the threads 80 of the distal end 25 of the handle 24, causing the illumination cartridge assembly 36 to move into contact with the contained batteries 70. Creating a negative electrical contact as the batteries 70 are pushed axially against the contact spring 62, with the strip and contact disk, the strip of the contact 129 contacts the cartridge collar 133 wherein the contact board 135 within the cartridge body 107 contacts the positive battery terminals, completing the circuit, and energizing the LED 108.

It should be noted that other forms of switch assemblies can be utilized, such as those that are described in U.S. Ser. No. 10/941,530, previously incorporated in its entirety herein.

In order for the user to properly sense actuation of the miniature LED 108, a spring member 144 is sandwiched, according to this embodiment, between the exterior of the internal cage member 50 and the interior of the handle 24. The spring member 144, in this instance, a leaf spring, is fastened at one end to a slot 54 of the internal cage member 50 adjacent the distal end 110 thereof by conventional means, in this case, by means of an interference fit, the spring member including a depending engagement end portion that initially resides in a groove which is provided at the distal end of the internal cage member. When the internal cage member 50 is inserted into the interior of the handle 24, the spring member 144 is biased such that the engagement end portion is caused to extend through a small opening (not shown) that is provided in the external threads 80 of the handle 24 that is sized to accommodate same.

In operation and referring to FIGS. 1-4, the support collet 84 is attached to the distal end 25 of the handle 24 by threading the internal threaded portion thereof with the external threads 80 provided on the distal end 25 of the handle 24. As the collet supporting ring 88 and support collet 84 are rotated thereupon in a clockwise manner to initiate engagement, the miniature LED 108 is caused to illuminate due to the above electrical connection that is created between the illumination cartridge assembly 36, including the contained LED 108 and the batteries 70. As the above procedure is reversed, the LED 108 is deenergized upon counterclockwise rotation of the collet supporting ring 88 and support collet 84 relative to the handle 24 of the instrument whereupon the engagement end portion is arranged relative to a hollow depression that is formed on the interior of the collet into which the engagement end portion repeatably slips when the LED OFF position is reached as mechanically sensed by the user in the form of a detent.

The disposable mirror 44 is first attached to the examination instrument 20 using colleted portions which are formed in the extending portion of the support collet 84, forming part of the front end cap 28 with the collet supporting ring 88. The front end cap 28 is threaded onto the distal end 25 of the handle 24 in order to secure the mirror 44 in place. The illumination cartridge assembly 36 is attached to the distal end of the handle 24 by threaded portions. In this position, the shank portion 96 of the mirror 44 is situated in relation to the miniature LED 108.

Emitted light from the miniature LED 108 is then focused, by virtue of the reflector cap portion onto the light transmissive shank portion 96 of the mirror 44. The mirror 44 thereby provides suitable illumination at the exit surfaces 99, 101 of the heel portion and the face portion, wherein the mirrored surface 103 permits enhanced examination of the teeth.

Following use, the mirror 44 can be removed by reversing the threading of the front end cap 28 and pulling same from the colleted areas of the handle 24. The front end cap 28 can then be removed, permitting access to the illumination cartridge assembly 36 can be removed by reversing the threaded portion of the handle and the distal cap from the body along with the batteries, once the mirror 44 has also been removed from the distal end of the examination instrument 20. Once removed, the handle 24, as well as the front and rear end caps 28, 32, can then be autoclaved.

To perform a photo-curing procedure, a similar assembly is initiated wherein illumination cartridge assembly 40, FIG. 2, is used in lieu of illumination cartridge assembly 36. As previously noted, the primary difference between these assemblies 36, 40 is the type of light source used. In this instance, illumination cartridge assembly 40 uses a blue LED such as Model LXHL-PB01, manufactured by Lumileds, Inc., which produces a wavelength of about 469 to 490 nanometers.

In lieu of the mirror 44, the transilluminator 48, such as a Model 5070, manufactured by Welch Allyn, Inc., is utilized, the transilluminator having an elongated shank portion which is inserted into the collet 88 of the front end cap 28 once the illumination cartridge assembly 40 has been added to the confines of the handle 24.

The transilluminator 48 includes a light emitting end 49 which conducts the blue light emitted by the contained cartridge assembly 40. The instrument is enabled by the switch assembly through rotation of the front end cap 28, as previously discussed, in order to initiate electrical engagement with the contained batteries 70, wherein the configuration of the cartridge body 107 prevents reverse polarity as previously described, and as shown in FIG. 8.

### PARTS LIST FOR FIGS. 1-8

20 dental instrument
24 elongated handle or body
25 open distal end
26 open proximal end
28 front end cap
32 rear end cap
36 illumination cartridge assembly
40 illumination cartridge assembly
44 mirror
48 transilluminator
49 light emitting end
50 interior cage member
54 lateral slots
58 electrically conductive battery contact strip
62 contact spring
66 proximal end
70 batteries
72 positive terminal
73 negative terminal
74 battery compartment
75 battery contact disk
80 screw threads
84 support collet
88 collet supporting ring
92 exterior knob
96 shank portion
97 light entrance surface
99 light exit surface
101 light exit surface
103 mirrored surface
107 cartridge body
108 miniature LED
110 distal end
114 reflector cap portion
118 PCB (printed circuit board)
122 opening
126 projecting portion
127 lateral slot
129 first electrical contact
130 heat sink
131 second electrical contact
133 cartridge collar
135 battery contact board
137 rivet
139 chamfered hole
144 ring member

Other variations and modifications will be readily apparent to one of sufficient skill in the field that embody the inventive concepts that are taught in the above specification and are herein recited in the following claims:

We claim:

1. A dental instrument comprising:
a handle:
a first light generating system releasably contained in said handle, said first light generating system including a first light source;
a first dental appliance releasably attachable to said handle and coupled to said first light generating system;
a second light generating system which can be substituted into said handle for said first light generating system, said second light generating system including a second light source which is different than the first light source.

2. A dental instrument as recited in claim 1, wherein each of said first and second light sources comprise at least one LED.

3. A dental instrument as recited in claim 1, including a second dental appliance which is releasably attachable to said handle and coupled to said second light generating system.

4. A dental instrument as recited in claim 2, wherein said first light source is at least one white LED and said second light source is at least one blue LED.

5. A dental instrument as recited in claim 1, wherein said first dental appliance is an intraoral dental mirror.

6. A dental instrument as recited in claim 3, wherein said second dental appliance is a dental curing element.

7. A dental instrument as recited in claim 6, wherein said dental curing element is a transilluminator.

8. A dental instrument as recited in claim 1, wherein said first dental appliance is disposable.

9. A dental instrument as recited in claim 3, wherein at least one of said first and second dental appliances is disposable.

10. A dental instrument as recited in claim 1, wherein said first and second light generating systems each comprise a cartridge containing one of said first and second light sources.

11. A dental instrument as recited in claim 10, wherein said cartridge is defined by a body which is releasably fitted within said handle.

12. A dental instrument as recited in claim 1, wherein said instrument is cordless.

13. A dental instrument as recited in claim 12, wherein said handle includes a battery compartment.

14. A dental instrument as recited in claim 1, wherein at least a portion of said instrument is autoclavable.

15. A dental instrument as recited in claim 10, wherein said cartridge includes at least one circuit board interconnected to said at least one retained light source.

16. A dental instrument as recited in claim 11, wherein said instrument includes a battery compartment adjacent said cartridge, said cartridge body being configured for preventing reverse battery polarity caused by at least one battery being improperly loaded into said battery compartment.

17. A dental instrument as recited in claim 16, wherein said cartridge body includes an opening extending to an interior electrical contact, said opening being sized for permitting engagement of said electrical contact with one type of battery terminal.

18. A method for performing separate dental procedures on a patient, said method comprising the steps of:
performing a first dental procedure on said patient using a dental instrument, said instrument having a first light source contained therein suitable for said first procedure;
releasably interchanging a second light source for said first light source in said instrument; and
performing a second dental procedure on said patient using said instrument.

19. A method as recited in claim 18, including the additional steps of:
coupling said first light source to a first dental appliance for performing said first procedure; and
coupling the second light source to a second light source for performing the second procedure.

20. A method as recited in claim 19, wherein each of said first and second light sources include at least one LED.

21. A method as recited in claim 19, wherein said first procedure is a visual examination of the teeth in which the first appliance is an intraoral dental mirror and the second procedure is a dental curing procedure.

22. A method as recited in claim 21, wherein said second appliance is a transiluminator releasably attached to said handle.

23. A method as recited in claim 18, wherein said dental instrument includes at least one battery as a power source, said method including the step of disabling operation of said instrument if said at least one battery is incorrectly loaded therein.

24. A method as recited in claim 18, wherein said instrument includes a first illumination cartridge containing said first light source and a second illumination cartridge containing said second light source.

25. A method as recited in claim 24, wherein said instrument is battery powered and in which each of said first and second illumination cartridges include a body which is configured to prevent reverse battery polarity.

26. A dental instrument comprising:
a handle;
a first light cartridge releasably insertable into said handle;
an intraoral dental mirror portion releasably attachable to said instrument and couplable to said first light cartridge such that light emitted from said cartridge is directed to said mirror portion; and
a second light cartridge interchangeably insertable into said handle in lieu of said first light cartridge, said first and second light cartridges having different light sources.

27. A portable medical instrument comprising:
a housing including a battery compartment;
an illumination system including at least one LED and circuitry for powering said at least one LED in order to provide illumination to a medical target, each of said at least one LED and said circuitry being provided within a cartridge body, said body being configured to prevent reverse battery polarity.

28. An instrument as recited in claim 27, wherein said cartridge body includes an opening extending to a contained electrical contact, said opening being sized to permit engagement only between said electrical contact and one type of terminal of a battery contained in said instrument.
